(19) United States
(12) Patent Application Publication
(10) Pub. No.: US 2020/0348245 A1
(43) Pub. Date: Nov. 5, 2020

(54) APPARATUS AND METHOD FOR INSECT INFESTATION DETECTION

(71) Applicant: KING FAHD UNIVERSITY OF PETROLEUM AND MINERALS, Dhahran (SA)

(72) Inventors: Sharif Iqbal Mitu SHEIKH, Dhahran (SA); Forhad HOSSAIN, Dhahran (SA)

(73) Assignee: KING FAHD UNIVERSITY OF PETROLEUM AND MINERALS, Dhahran (SA)

(21) Appl. No.: 16/400,252

(22) Filed: May 1, 2019

(51) Int. Cl. G01N 22/00; G01N 33/00

(52) U.S. Cl. CPC G01N 22/00; G01N 33/0098

(57) ABSTRACT

An apparatus and a method of detecting insect infestation condition are described. The apparatus can include: a microstrip patch antenna, configured to mount on a flattened surface of a test tree trunk and transmit a microwave signal; a measurement device, configured to measure a microwave reflection response of the test tree trunk in response to the microwave signal from the microstrip patch antenna; a storage medium, configured to store the measured microwave reflection response of the test tree trunk, one or more microwave reflection responses of one or more reference tree trunks, a program for comparing microwave reflection responses, and a program for identifying an insect infestation condition of the test tree trunk, and a processing circuitry, configured to read the stored microwave reflection responses, compare the measured microwave reflection response of the test tree trunk to the microwave reflection responses of one or more reference tree trunks, and identify the insect infestation condition of the test tree trunk.

### BACKGROUND OF THE INVENTION

### Field of the Invention

The present disclosure relates to an apparatus and a method for insect infestation detection, and specifically relates to Red Palm Weevil (RPW) infestation in palm trees.

### Description of the Related Art

Red Palm Weevil (RPW), with a scientific name of Rhynchophorus Ferrungineus, is the most disruptive and widespread insect of the palm trees. Early detection of RPW infestation is critical to save the tree and stop the insect from infecting the neighboring trees of the plantation. Several approaches have been used to detect the RPW infestation in palm trees. Visual inspection technique is a popular approach to detect RPW infestation. However, the visual inspection technique cannot detect RPW infestation in an early stage due to the hidden larva amid leaf bases or stem fibers (see V. A. Abraham, et al, “An Integrated Management Approach for Red Palm Weevil Rhynchophorus Ferrugineus Oliv. a Key Pest of Date Palm in the Middle East.” Journal oaf Agricultural and Marine Sciences [S. I.], v. 3, n. 1, p. 77-83, January 1998, incorporated herein by reference in its entirety).

Another approach commonly used is chemical detection, where fermenting odor emitting from the wounds in infested palm trees can be picked up by well-trained sniffing dogs (see V. Soroker et al., “Early Detection and Monitoring of Red Palm Weevil: Approaches and Challenges,” Association Française de Protection des Plantes (AFPP) Colloque méditerranéen sur les ravageurs des palmiers, Nice, France, 16-18 Jan. 2013, incorporated herein by reference in its entirety). In this widely used technique, detection also comes in a later stage of RPW infestation.

In recent years, acoustic and thermal imaging techniques are becoming popular in detecting early stage infestation of the RPW (see N. Al-Dosary, et al, “Review on the Management of Red Palm Weevil Rhynchophorus Ferrugineus Olivier in Date Palm Phoenix Dactylifera L,” Emirates Journal of Food and Agriculture, Vol. 28, no. 1, pp. 34-44, December 2015; V. Soroker et al., “Early Detection and Monitoring of Red Palm Weevil: Approaches and Challenges,” Association Francaise de Protection des Plantes (AFPP) Colloque méditerranéen sur les ravageurs des palmiers, Nice, France, 16-18 Jan. 2013, and R. Massa et al., “Experimental and numerical evaluations on palm microwave heating for Red Palm Weevil pest control,” Nat. Publ. Gr., no. March, pp. 1-8, 2017, each incorporated herein by reference in their entirety). But the acoustic and thermal data measured in the early stage of the infestation are strongly affected by the noise presented in nature.

Infrared cameras have recently been used to detect the change in tree temperature due to the RPW infestation. The main reason of the temperature increase is intensive fermentation within the tree trunk, which often exceeds 45° C. M. Mozib et al. used a real-time temperature sensor to detect infested palm trees (see M. Mozib and H. A. El-Shafie, “Effect of Red Palm Weevil, Rhynchophorus Ferrugineus (Olivier) Infestation on Temperature Profiles of Date Palm Tree,” J. Entomol. Nematol., vol. 5, no. 6, pp. 77-83, 2013, incorporated herein by reference in its entirety). But this method is strongly influenced by the weather conditions surrounding the tree.

Dielectric resonators that employ microwave, parallel plate capacitor, and transmission line methods are popular in characterizing material properties (see M. Taha, W. Peng, M. Zaka, and U. Rehman, “Microwave sensor for nondestructive dielectric characterization of biological systems,” Int. J. Appl. Electromagn. Mech., vol. 50, pp. 353-363, 2016; M. S. Boybay and O. M. Ramahi, “Material Characterization Using Complementary Split-Ring Resonators,” IEEE Trans. Instrum. Meas., vol. 61, no. 11, pp. 3039-3046, 2012; A. K. Verma, et al., “Microstrip resonator sensors for determination of complex permittivity of materials in sheet, liquid and paste forms,” IEE Proceedings-Microwaves, Antennas Propag., vol. 152, no. 1, pp. 47-54, 2005; R. T. Sheldon, “Radiofrequency and capacitive sensors for dielectric characterization of low-conductivity media,” 2015; and M. D. Janezic and. D. F. Williams, “Permittivity characterization from transmission line measurement,” in Microwave Symposium Digest, IEEE MTT-S International, vol. 3, pp. 1343-1346. 1997, each incorporated herein by reference in their entirety). Conformal microstrip patch antennas are widely used to excite resonant cavities, where dielectric properties are approximated by monitoring the changes in antenna impedance and resonance frequency (see Y. Li, S. Member, N. Bowler, S. Member, and D. B. Johnson, “A Resonant Microstrip patch Sensor for Detection of Layer Thickness or Permittivity Variations in Multilayered Dielectric Structures,” IEEE Sensors Journal, vol. 11, no. 1, pp. 5-15, 2011 and C. Yang, C. Lee, A. Member, K. Chen, S. Member, and K. Chen, “Noncontact Measurement of Complex Permittivity and Thickness by Using Planar Resonators,” IEEE Trans, Microw. Theory Tech., vol. 64, no. 1, pp. 247-257, 2016, each incorporated herein by reference in their entirety). Based on this observation inventors disclose herein a method and system that applies this technique to monitor RPW infestation by linking the resonant frequency with the dielectric properties of the tree trunk.

In the present disclosure, an apparatus that includes a 1-GHz circular patch antenna is placed in contact with a tree trunk to monitor the dielectric properties that are related to RPW infestation in the tree trunk. The apparatus can be integrated with existing microwave treatment systems wherein a high-power microwave signal is used to kill the larva within an RPW infested tree. This technique is especially effective in minimizing the spread of the insects during the uprooting and discarding of the damaged palm trees from the plantation.

### SUMMARY

Aspects of the disclosure provide an apparatus and a method for detecting insect infestation in palm trees. The apparatus can include: a microstrip patch antenna, configured to mount on a surface of a test tree trunk and transmit a microwave signal; a measurement device, configured to measure a microwave reflection response of the test tree trunk: a storage medium, configured to store the measured microwave reflection response of the test tree trunk, microwave reflection responses of one or more reference tree trunks, a program for comparing microwave reflection responses, and a program for identifying an insect infestation condition of the test tree trunk; and a processing circuitry, configured to read the stored microwave reflection responses, compare the measured microwave reflection response of the test tree trunk to the microwave reflection responses of one or more reference tree trunks, and identify the insect infestation condition of the test tree trunk.

In an embodiment, the test tree trunk is a superstrate of the microstrip patch antenna.

In an embodiment, the microstrip patch antenna can be configured to place the circular patch of the microstrip patch antenna in contact with a manually flattened surface of the test tree trunk wherein the size of flattened surface is not smaller than the size of microstrip patch antenna.

In an embodiment, when transmitting the microwave signal from the microstrip patch antenna, the microstrip patch antenna is further configured to transmit the microwave signal from the microstrip patch antenna at a microwave signal frequency not above 1.2 GHz.

In an embodiment, when storing one or more microwave reflection responses of one or more reference tree trunks, the storage medium can be further configured to store simulated microwave reflection responses of one or more reference tree trunks under different insect infestation conditions including healthy, partially damaged, or/and completely damaged, or measured microwave reflection responses of one or more reference tree trunks under different insect infestation conditions including healthy, partially damaged, or/and completely damaged, wherein the insect infestation conditions are identified beforehand.

In an embodiment, when comparing the measured microwave reflection response of the test tree trunk to the microwave reflection responses of one or more reference tree trunks, the processing circuitry can further configured to load the program, stored in the storage device, for comparing the measured microwave reflection response of the test tree trunk to the microwave reflection responses of the one or more reference tree trunks and execute the program to calculate the change between a resonant frequency of the test tree trunk and a resonant frequency of a healthy reference tree trunk, a partially damaged reference tree trunk, or/and a completely damaged reference tree trunk.

In another embodiment, when identifying the insect infestation condition of the test tree trunk, the processing circuitry is further configured to identify the test tree trunk to be a healthy tree trunk when the change between a resonant frequency of the test tree trunk and a resonant frequency of a healthy reference tree trunk is below a threshold value, identify the test tree trunk to be a partially damaged tree trunk when the change between a resonant frequency of the test tree trunk and a resonant frequency of a partially damaged reference tree trunk is below the threshold value, or identify the test tree trunk to be a completed damaged tree trunk when the change between a resonant frequency of the test tree trunk and a resonant frequency of a completed damaged reference tree trunk is below the threshold value.

Aspects of the disclosure provide a method of detecting insect infestation in palm trees. The method can comprise mounting a microstrip patch antenna on a manually flattened surface of a test tree trunk; transmitting a microwave signal from the microstrip patch antenna; measuring, by a measurement device, a microwave reflection response of the test tree trunk; storing, by a storage medium, the measured microwave reflection response of the test tree trunk, microwave reflection responses of one or more reference tree trunks, a program for comparing microwave reflection responses, and a program for identifying an insect infestation condition of the test tree trunk; and reading, by a processing circuitry, the stored microwave reflection responses; comparing, by the processing circuitry, the measured microwave reflection response of the test tree trunk to the microwave reflection responses of one or more reference tree trunks; and identifying, by the processing circuitry, the insect infestation condition of the test tree trunk.

Aspects of the disclosure further provide a non-transitory computer readable medium storing instructions which, when executed by a processing circuitry, cause the processing circuitry to perform a method for reading stored microwave reflection responses, comparing a measured microwave reflection response of a test tree trunk to microwave reflection responses of one or more reference tree trunks, and identifying an insect infestation condition of the test tree trunk.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows an exemplary microstrip patch antenna according to an embodiment of the disclosure;

FIG. 2 is an exemplary table for dielectric properties of tree trunk under different insect infestation conditions according to an embodiment of the disclosure;

FIG. 3 shows an experimental simulated reflection response of the tree trunks under different insect infestation conditions according to an embodiment of the disclosure;

FIG. 4 shows an exemplary experimental setup according to an embodiment of the disclosure;

FIG. 5 shows an experimental reflection response of the tree trunks under, different insect infestation conditions according to an embodiment of the disclosure; and

FIG. 6 is a flowchart outlining an exemplary process of detecting insect infestation according to an embodiment of the disclosure.

### DETAILED DESCRIPTION OF THE EMBODIMENTS

An apparatus and a method for detecting insect infestation are described in the present disclosure. The apparatus and the method can detect an insect infestation at an early stage without damaging the tree and identify the infestation conditions of the tree trunk. The apparatus can further be integrated with existing microwave treatment systems (see Massa, R., Caprio, E., De Santis, M., Griffo, R., Migliore, Panariello, G., Pinchera, D. and Spigno, P., “Microwave treatment for pest control: the case of Rhynchophorus ferrugineus in Phoenix canariensis,” EPPO Bulletin, 41(2), pp.128-135, 2011, incorporated herein by reference in its entirety). Specifically, the apparatus includes a microstrip patch antenna, which includes or consists of a circular patch, an antenna substrate, an optional cable connector such as a coaxial cable connector, and an antenna ground (GND) plane. The microstrip patch antenna can be configured to mount on a flattened surface of a test tree trunk and transmit a microwave signal. A measurement device is configured to measure a microwave reflection response of the test tree trunk and store the measured microwave reflection response of the test tree trunk into a storage medium. The storage medium can be further configured to store microwave reflection responses of one or more reference tree trunks, a program for comparing microwave reflection responses, and a program for identifying an insect infestation condition of the test tree trunk. Processing circuitry is then configured to read, the stored microwave reflection responses and compare the measured microwave reflection response of the test tree trunk to the microwave reflection responses of one or more reference tree trunks. Based on the comparison results, the processing circuitry can identify the insect infestation condition of the test tree trunk.

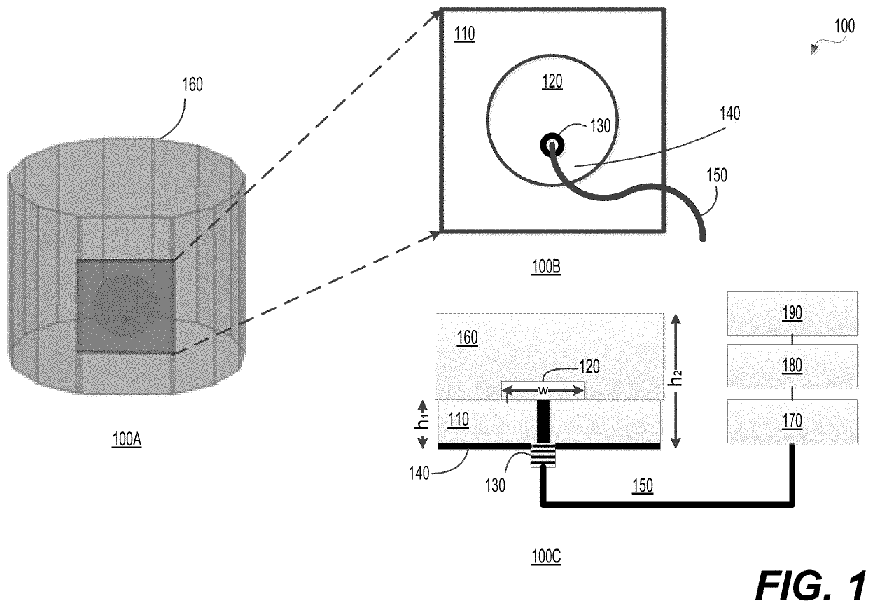

FIG. 1 shows an exemplary microstrip patch antenna 100 according to an embodiment of the disclosure. In the FIG. 1 example, the microstrip patch antenna 100 can be illustrated by a 3-dimentional (3-D) schematic view 100A, a top view 100B, and a schematic view 100C showing options for the interconnectivity of components of the microstrip patch antenna.

As shown in the 3-D schematic view 100A of the FIG. 1, a microstrip patch antenna 100 can be mounted on a tree trunk 160 and the tree trunk 160 can act as a superstrate of the microstrip patch antenna 100. Specifically. an area of the surface of the tree trunk 160 can be manually changed by a tool to be a flat base, so that the microstrip patch antenna 100 can be placed in contact with the flattened area (i.e., the flat base) of the tree trunk to avoid air-gaps between the microstrip patch antenna 100 and the tree trunk 160. The microstrip patch antenna 100 can be further stabilized on the flat base of the tree trunk 160 by any means, such as a paste spread on the trunk, a tape wrapping around the tree trunk 160 and the microstrip patch antenna 100, and the like. In other embodiments the surface of the tree trunk can be smoothed but curved. The microstrip patch antenna may have a corresponding curved surface that matches or closely matches the surface of the tree trunk. In still other embodiments a transmittance medium may be applied to the surface of the tree trunk (smoothed or in its naturally occurring state) so that the microstrip patch antenna is in direct contact with the transmittance medium and/or tree trunk without any air gaps or void. The transmittance medium may be a solid moldable material, a gel, a compressible material or a liquid material that cures to form a solid support. Preferably the transmittance medium has a microwave transmittance that is similar to the microwave transmittance of the tree trunk.

In some embodiments, a plurality of the microstrip patch antennas 100 can be mounted around the tree trunk 160 so that electromagnetic (EM) signals excited from the microstrip patch antenna 100 can penetrate the tree trunk 160.

The tree trunk 160 can be a healthy tree trunk, a partially damaged tree trunk by insect infestation, and a completely damaged tree trunk by insect infestation. It should be understood that any portion of any species of a tree can be selected as the superstrate of the microstrip patch antenna 100. The tree trunk 160 can represent a test tree trunk or one or more reference tree trunks. In the present disclosure, the tree trunk 160 can be a date palm tree trunk, with a height which is at least 5-10 cm larger than the height of the microstrip patch antenna 100. Damage in the tree trunk may be in the form of voids or partially filled voids. For example, the presence of an insect infestation in the tree trunk may be evident from the damage caused by the insects, e.g., voids and/or voids that are filled with degraded material. The presence of an infestation may also be evident from the presence of insects. Voids and partially filled voids inside a tree trunk produce a different microwave signal and/or are detectable through a different microwave penetration, permittivity or propagation through the tree trunk in comparison to a healthy tree trunk that, is not infested with insects.

As shown in the top view 100B and the side view 100C of the FIG. 1 example, the microstrip patch antenna 100 can include an antenna substrate 110, a circular patch 120, a cable connector 130 such as a coaxial cable connector, and an antenna ground (GND) plane 140. A transmission cable 150 can connect to the microstrip patch antenna 100 through the cable connector 130 and feed through the antenna GND plane 140. Herein, the antenna substrate 110 can be any insulating dielectric substrate, such as a printed circuit board (PCB), with a continuous metal layer bonded to the opposite side of the substrate which forms the antenna GND plane 140. The thickness of the antenna substrate 110 can vary from 0.05 mm to 100 min, preferably from 0.1 to 50 mm, 0.5 to 40 mm, 1 to 30 mm, 2 to 20 mm, 3 to 10 mm, 4 to 8 mm or about 5 mm. A thicker substrate increases the gain of the microstrip patch antenna 100 to some extent, but may lead to undesired effects like surface wave excitation: surface waves decrease efficiency and perturb the radiation pattern. The antenna GND plane 140 can extend beyond the edges of the antenna substrate 110 by at least 2 to 3 times the substrate thickness for proper operation. An antenna GND plane 140 that is too small can result in a reduced front to back ratio. Making the antenna GND plane 140 larger can also increase the gain of the microstrip patch antenna 100, but as the antenna GND plane 140 size increases, diffraction near the edges can play less of a role and increasing the size of an already “large” ground plane has very little effect on gain.

The circular patch 120 can be a patch of metal foil which is fabricated by etching the metal elements pattern in the antenna substrate 110. It should be understood that any shape of the metal foil can be used to form a microstrip antenna.

The transmission cable 150 can be a specialized cable or other structure designed to conduct alternating current of radio frequency. In the FIG. 1 example, the transmission cable 150 can be a coaxial cable that is connected to the cable connector 130, wherein the outer conductor of the one end of the cable is connected to the antenna GND plane 140, and the center conductor of the one end of the cable is extended up to the circular patch 120. The other end of the cable can connect to a measurement device 170, such as a network analyzer and an oscilloscope. Herein, the measurement device 170 is a stable power radio frequency (RF) generator which can provide a stable power to the microstrip patch antenna 100 and generate EM signals with a configured frequency. Usually, the power level of the measurement device 170 can be from a fraction of one watt to a few watts, and the frequency of the generated EM signals can vary from 600 MHz to 2 GHz. In the present disclosure, the frequency is preferably in the range of from 0.6 to 1.2 GHz, more preferably from 0.8 to 1 GHz or about 0.9 GHz.

The measurement device 170 can further measure a reflection responses of exciting a material or an object which the microstrip patch antenna 100 is mounted on, and stored the measurement data in a storage medium 180. The storage medium 180 can be any device or material that can place, keep and retrieve electronic data, such as operating systems, application programs, measured reflection response data of trees, and the like. It can include a read only memory (ROM), a random access memory (RAM), a flash memory, a solid state memory, a hard disk drive, an optical disk drive, and the like.

Herein, a reflection response records a sequence of response amplitudes that an object or a material oscillates in a specific frequency. In particular, a frequency at which the response amplitude is a relative maximum is known as a resonant frequency. The resonant frequency is one of the dielectric properties and is closely related to the material and internal structure of an object.

In the present disclosure, the storage medium 180 can store the measured microwave reflection response of the test tree trunk and microwave reflection responses of one or more reference tree trunks, such as a healthy tree trunk's microwave reflection response, a partially damaged tree trunk's microwave reflection response, or/and a completely damaged tree trunk's microwave reflection response. In addition, the storage medium 180 can store programs for comparing the measured microwave reflection response of the test tree trunk to the stored microwave reflection responses of the tree trunks, and identifying an insect infestation condition of the test tree trunk.

Further, a processing circuitry 190 is used to read the stored microwave reflection responses, and execute the aforementioned programs to detect resonant frequencies from the microwave reflection responses of the tree trunks, which include both the test tree trunk and the reference tree trunks. The processing circuitry 190 can then compare the resonant frequency of the test tree trunk to that of the reference tree trunks with different insect infestation conditions. Based on the comparison results, the processing circuitry 190 can identify whether the test tree trunk is infected or not, and identify the insect infestation condition accordingly.

The equation that is used to calculate a resonant frequency of a microstrip patch antenna 100 with a tree trunk 160 as a superstrate can be expressed as formula (1) (see R. Kumar And P. Malathi, “Effects of Superstrates on The Resonant Frequency of Rectangular. Microstrip Antennas.” Microwave and Optical Technology Letters, vol. 49, no. 12, pp. 2946-2950, 2007 and S. Zhong, G. Liu, and G. Qasim, “Closed-Form Expressions for Resonant Frequency of Rectangular Patch Antennas with Multi-dielectric Layers,” IEEE Trans. on Antenna and Propagation, vol. 42, no. 9, pp. 1360-1363, 1994, each incorporated herein by reference in their entirety)

$${f}_{r} = \frac{1.8412 * c}{2 π {a ( {ɛ}_{eff} ( f ) )}^{\frac{1}{2}}} , \qquad ( 1 )$$

where, c is the speed of light, α is the radius of the circular patch 120 and ε_{eff}(f) is the effective dielectric constant of the microstrip patch antenna 100 with the tree trunk 160. Herein, the microstrip patch antenna 100 with the tree trunk 160 can have a multilayer structure including covered dielectric materials, tree trunk thickness, or an unintentional air-gap between the circular patch 120 and the tree trunk 160. Therefore, the effective dielectric constant ε_{eff}(f) of the multilayer structured rnicrostrip patch antenna 100 with the tree trunk 160 as the superstrate can be obtained by calculating a frequency independent dielectric constant ε_{eff}(0) of the microstrip patch antenna 100 with the tree trunk 160 as the superstate, which can be given by:

$$\begin{matrix}{ɛ}_{eff} ( f ) = {ɛ}_{r}^{′} - \frac{{ɛ}_{r}^{′} - {ɛ}_{eff} ( 0 )}{1 + P ( f )} , & ( 2 ) \\ {ɛ}_{eff} ( 0 ) = {q}_{1} + {q}_{2} + {ɛ}_{2} \frac{{( 1 - {q}_{1} - {q}_{2} )}^{2}}{{ɛ}_{2} ( 1 - {q}_{1} - {q}_{2} - {q}_{3} ) + {q}_{3}} . & ( 3 )\end{matrix}$$

Here, ε′ᵣ is an equivalent relative permittivity. q₁, q₂, and q₃ are three filing factors that represent three different dielectric layers, which can be expressed as:

$$\begin{matrix}{q}_{1} = \frac{1}{2} \{ 1 + \frac{π}{4} + \frac{{h}_{1}}{{w}_{e}} \times ln [ \frac{2 {w}_{e}}{{h}_{1}} sin ( \frac{π}{2} ) ] \} & ( 4 ) \\ {q}_{2} = 1 - {q}_{1} - \frac{{h}_{1}}{2 {w}_{e}} ln ( \frac{π {w}_{e}}{{h}_{1}} - 1 ) & ( 5 ) \\ {q}_{3} = 1 - {q}_{1} - {q}_{2} - \frac{{h}_{1} - {v}_{e}}{2 {w}_{e}} \times ln [ \frac{2 {w}_{e}}{2 {h}_{13} - {h}_{1} + {v}_{e}} \cdot cos ( \frac{π {v}_{e}}{2 {h}_{1}} ) + sin ( \frac{π {v}_{e}}{2 {h}_{1}} ) ] & ( 6 ) \\ {w}_{e} = w + \frac{2 {h}_{1}}{π} ln [ 17 \cdot .08 ( \frac{w}{2 {h}_{1}} + 0.92 ) ] & ( 7 ) \\ {v}_{e} = 2 \frac{{h}_{1}}{π} arctan [ \frac{π}{\frac{π}{2} \frac{{W}_{e}}{{h}_{1}} - 2} ( \frac{{h}_{1}}{{h}_{2}} - 1 ) ] & ( 8 ) \\ {ɛ}_{r}^{′} = \frac{2 {ɛ}_{eff} ( 0 ) - 1 + A}{1 + A} & ( 9 ) \\ A = {( 1 + \frac{12 {h}_{1}}{w} )}^{- \frac{1}{2}} & ( 10 ) \\ P ( f ) = {P}_{1} {{P}_{2} [ ( 0.1844 + {P}_{3} {P}_{4} ) 10 fh ]}^{1.5763} & ( 11 ) \\ {P}_{1} = 0.27488 + [ 0.6315 + 0.525 / {( 1 + 0.157 fh )}^{20} ] \cdot u - 0.065683 exp ( - 8.7513 u ) & ( 12 ) \\ {P}_{2} = 0.33622 [ 1 - exp ( - 0.03442 {ɛ}_{r} ) ] & ( 13 ) \\ {P}_{3} = 0.0363 exp ( - 4.6 u ) \times \{ 1 - exp [ - {( fh / 3.87 )}^{4.97} ] \} & ( 14 ) \\ {P}_{4} = 1 + 2.751 \{ 1 - exp [ - {( {ɛ}_{r} / 15.916 )}^{8} ] \} & ( 15 )\end{matrix}$$

where h₁ is the thickness of antenna substrate 110, h₂ is the sum of the thickness of antenna substrate 110 and the thickness of the tree trunk 160 as the superstrate, u=w/h₁ denotes the circular patch 120 width (diameter) normalized with respect to the thickness of the antenna substrate 110 and fh is the normalized frequency with respect to free space wavelength (note: fh˜h/λ₀ where λ₀ is the free space wavelength).

The radius of the circular patch antenna can be set to 0.5-10 cm, preferably from 1 to 8 cm, 2 to 6 cm, or about 5 cm. In the present disclosure, the radius of the optimized coaxial feed circular patch antenna can preferably be set to about 4 cm. In addition, Rogers TMM 6 materials with εᵣ and t=3.8 mm are preferably used as the antenna substrate. The tree trunk of a date palm tree can be the superstrate wherein the tree trunk can be completely damaged, partially damaged or healthy. The damage can be caused by an RPW infestation.

In operation, the microstrip patch antenna 100 can be mounted on a manually flattened surface of the test tree trunk 160 and/or the naturally curved surface of the tree trunk, optionally smoothed, and the test tree trunk 160 is the superstrate of the microstrip patch antenna 100. Further, the connector cable 150 can connect the microstrip patch antenna 100 with a measurement device (e.g., an oscilloscope) through the cable connector 130 and feed through the antenna GND plane 140.

The microstrip patch antenna 100 can then transmit microwave signals in different frequencies, e.g., separately as individual frequencies or narrow bands, or alternately as a combination of frequencies in a broad range. For example, as shown in the FIG. 1, the microstrip patch antenna 100 can excite the superstrate which is the test tree trunk 160, with a frequency ranging from 0.6 to 1.2 GHz and the increment step size can be set to 0.03 GHz. At each frequency, a microwave reflection response can be collected and measured by the measurement device 170 and stored in the storage medium 180.

Further, the processing circuitry 190 can compare the measured microwave reflection response of the test tree trunk 160 to the microwave reflection responses, which can be stored in the storage medium 180, of one or more reference tree trunks, and identify the insect infestation condition of the test tree trunk 160.

The tree trunk 160 in different insect infestation conditions can have different dielectric properties. such as different resonant frequencies, different relative permittivity values, different loss tangent values, and different conductivity values. By linking the measured microwave reflection response with the dielectric properties (e.g., resonant frequency, relative permittivity, loss tangent, and conductivity) of the tree trunk, the insect infestation condition of the tree trunk can be identified accordingly.

In some embodiments, the microwave reflection response of the one or more reference tree trunks can be simulated microwave reflection responses of a tree trunk in different insect infestation conditions: healthy, partially damaged, and completely damaged. The simulated microwave reflection responses can be generated by simulation software (e.g., HFSS) and stored in the storage medium 180.

In some other examples, the microwave reflection responses of the one or more reference tree trunks can be the measured microwave reflection responses of one or more reference tree trunks in different insect infestation conditions, and the insect infestation conditions can be known beforehand. Then, the one or more reference tree trunks in different insect infestation conditions can be measured and the measured microwave reflection responses can be stored in the storage medium 180 for comparison purpose. The test tree trunk 160 can compare its measured microwave reflection response with the simulated microwave reflection responses, or/and the measured microwave reflection responses of the one or more reference tree trunks in different insect infestation conditions.

From the measured microwave reflection response, the resonant frequency of the test tree trunk can be located and compared to identify the insect infestation condition of the test tree trunk 160. For example, when the difference between the resonant frequency of the test tree trunk 160 and the simulated resonant frequency of a healthy reference tree trunk is below a preset threshold (e.g., 10%, 5%, 2% or 1%), the test tree trunk 160 can be identified to be a healthy (e.g., un-infested) tree trunk. Similarly, when the different between the resonant frequency of the test tree trunk 160 and the simulated resonant frequency of a reference completely damaged tree trunk is below the preset threshold (e.g., 10%), the test tree trunk 160 can be identified to be a completely damaged tree trunk. When the different between the resonant frequency of the test tree trunk 160 and the simulated resonant frequency of a reference partially damaged tree trunk is below a preset threshold (e.g., 10%), the test tree trunk 160 can be identified to be a partially damaged tree trunk.

In some other embodiments, when the simulated microwave reflection responses of one or more reference tree trunks under different insect infestation conditions are not available, the measured resonant frequency of the test tree trunk 160 can be compared with a measured resonant frequency of a reference tree trunk whose insect infestation condition is known beforehand. For example, when the reference tree trunk is a healthy tree trunk and its microwave reflection response has been measured and stored in the storage medium 180 if the different between the resonant frequency of the test tree trunk 160 and the resonant frequency of the reference healthy tree trunk is below a preset threshold (e.g., 10%, 5%, 2% or 1%), then the test tree trunk 160 can be identified to be a healthy tree trunk. Otherwise, the test tree trunk can be identified to be a damaged tree trunk. Further, the insect infestation condition of the test tree trunk 160 can be evaluated based on the comparison with reference tree trunks that have different insect infestation conditions. For example, when the reference tree trunk is a completely damaged tree trunk and its microwave reflection response has been measured and stored in the storage medium 180, if the difference between the resonant frequency of the test tree trunk 160 and the resonant frequency of the reference completely damaged tree trunk is below the preset threshold (e.g., 10%, 5%, 2% or 1%), the test tree trunk 160 can be identified to be a completely damaged tree trunk. Similarly, when the reference tree trunk is a partially damaged tree trunk and its microwave reflection response has been measured and stored in the storage medium 180, if the difference between the resonant frequency of the test tree trunk 160 and the resonant frequency of the reference partially damaged tree trunk is below the preset threshold (e.g., 10%, 5%, 2% or 1%), the test tree trunk 160 can be identified to be a partially damaged tree trunk.

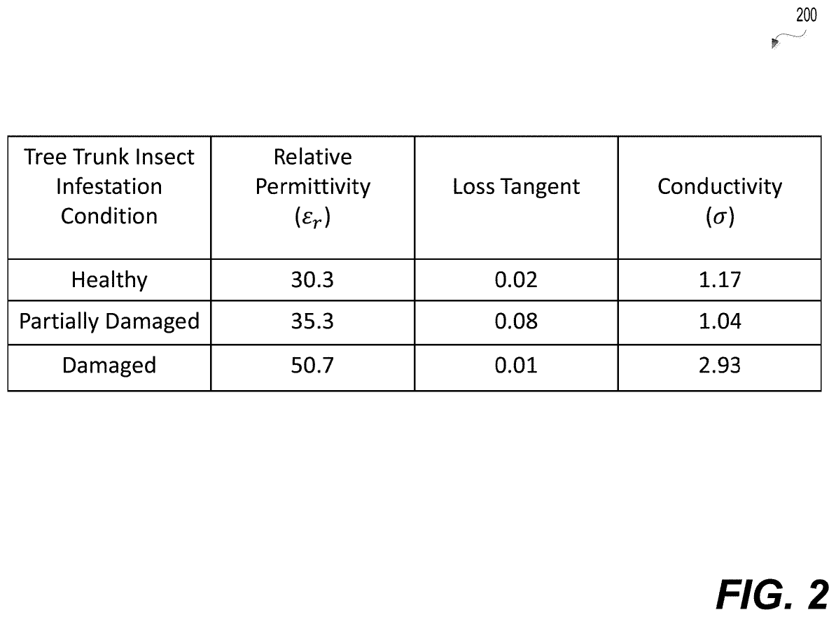

FIG. 2 lists an exemplary table 200 for dielectric properties of tree trunk under different insect infestation conditions according to an embodiment of the disclosure (see R. Massa, et al “Microwave treatment for pest control: the case of Rhynchophorus ferrugineus in Phoenix canariensis,” EPPO Bulletin, 41(2), pp.128-135, 2011, incorporated herein by reference in its entirety). Three tree trunks under different insect infestation conditions are included in the table: a healthy tree trunk, a partially damaged tree trunk, and a completely damaged tree trunk. Three types of dielectric properties are listed. Relative permittivity indicates how easily a material can become polarized by imposition of an electric field on an insulator. Loss tangent is defined by the angle between the capacitor's impedance vector and the negative reactive axis. Conductivity represents a material's ability to conduct electric current.

In the FIG. 2 example, a healthy tree trunk has a lowest relative permittivity, εᵣ=30.3. The relative permittivity of a tree trunk increases as the insect infestation condition becomes worse. For example, a partially damaged tree trunk has a relatively higher relative permittivity εᵣ=35.3, and a completely damaged tree trunk has a highest relative permittivity εᵣ=50.7.

For the loss tangent, the completely damaged tree trunk has a lowest value of σ=0.01. The healthy tree trunk has a relatively higher value of 0.02 and the partially damaged tree trunk has the highest value of 0.08.

For conductivity, the partially damaged tree trunk has a lowest value σ=1.04. The healthy tree trunk has a relatively higher value σ=1.17 and the partially damaged tree trunk has the highest value σ=2.93.

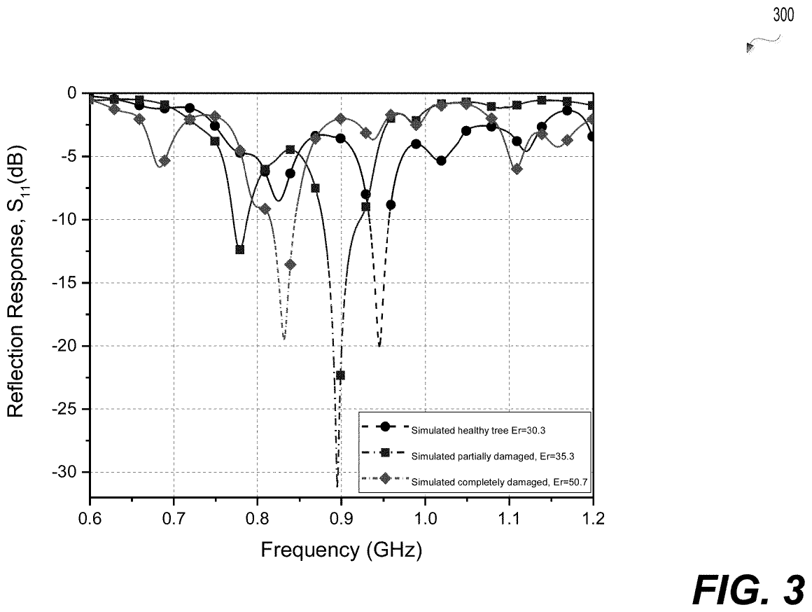

FIG. 3 shows a simulated reflection response (S₁₁) 300 of the microstrip patch antenna under different insect infestation conditions according to an embodiment of the disclosure. In the FIG. 3 example, the simulation can be performed on three palm tree trunks in three different RPW infestation conditions: healthy, partially damaged, and (completed) damaged. Professional software such as high-frequency structure simulator (HFSS) can be used to simulate the microwave reflection response (S₁₁) 300 on the three palm tree trunk.

With the increased RPW infestation, the wet oozing discharge from the infected part of the tree trunk increases the effective dielectric constant and reduces the resonant frequency. As shown in the FIG. 3, the resonant frequency of the healthy tree trunk is about 0.94 GHz and the resonant frequency of the completely damaged tree trunk is about 0.83 GHz. Therefore, a 12%

$$( i . e . , \frac{0.94 - 0.85}{0.94} \times 100 \% = 12 \% )$$

change in the resonance frequencies can be observed between the healthy and the damaged tree trunks.

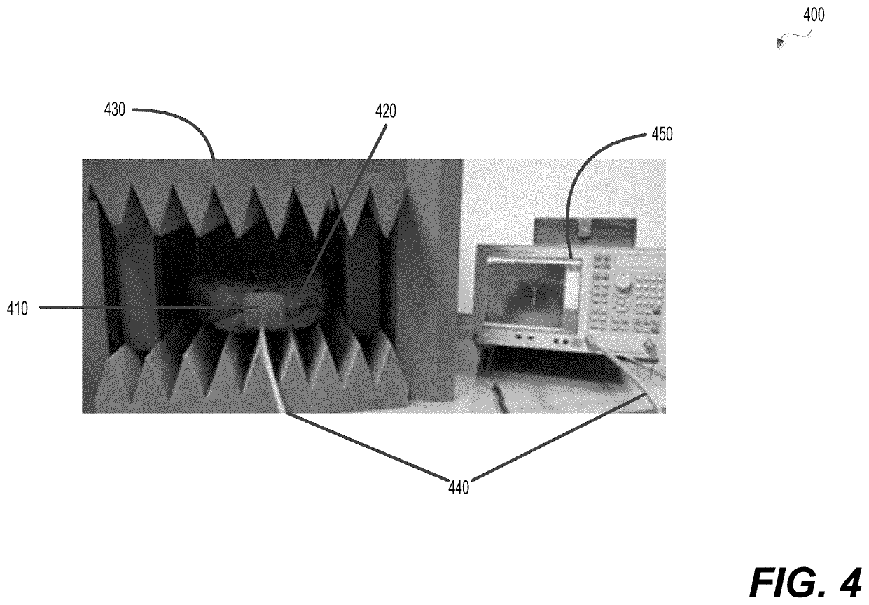

FIG. 4 shows an exemplary experimental setup 400 according to an embodiment of the disclosure. The experimental setup can be used to verify the simulated resonant behaviors of the microstrip patch antenna.

In the FIG. 4 example, the microstrip patch antenna 410 can be mounted on a manually flattened base of a test tree trunk 420 to avoid air-gaps between the microstrip patch antenna 410 and the test tree trunk 420. Herein, the test tree trunk 420 can act as the superstrate of the microstrip patch antenna 410. The microstrip patch antenna 410 together with the test tree trunk 420 can be placed in a test chamber (e.g., an anechoic chamber) 430 for dielectric measurement.

Further, a cable 440 can connect the microstrip patch antenna 410 with a measurement device (e.g., an oscilloscope) 450 through a connector and feed through the antenna GND plane of the microstrip patch antenna 410. The measurement device 450 can be a stable power radio frequency RF generator (e.g., an oscilloscope) which can provide a stable power to the microstrip patch antenna 410 and generate EM waves with a configured frequency. The configured frequency of the generated EM signals can vary from 0.6 GHz to 1.2 GHz, preferably 0.8 to 1.0 GHz or about 0.9 GHz with an increment step size of 0.03 GHz, alternately 0.1, 0.05, 0.02 or 0.01 GHz. The measurement device 450 can further measure the microwave reflection responses and store the measured microwave reflection responses in a storage medium, which is not displayed in the FIG. 4. By comparing the measured microwave reflection responses of the test tree trunk 420 to the microwave reflection responses, stored in the storage medium, of one or more reference tree trunks in different RPW infestation conditions, the RPW infestation condition of the test tree trunk 420 can be identified.

In an embodiment, because a single microstrip patch antenna 410 cannot penetrate the whole test tree trunk 420 without a high power microwave excitation signal, multiple low power microstrip patch antennas 410 can be mounted around the test tree trunk 420 for a high resolution measurement of the RPW infestation.

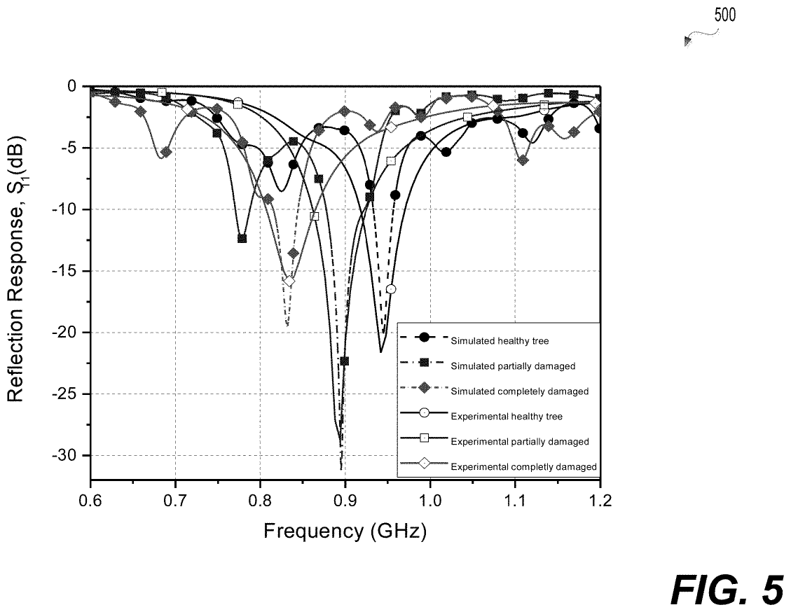

FIG. 5 shows an experimental reflection response 500 of the microstrip patch antenna under different insect infestation conditions according to an embodiment of the disclosure. In the FIG. 5 example, the simulation is performed on three test palm tree trunks in three different RPW infestation conditions: healthy, partially damaged, and completely damaged.

By comparing the experimental reflection response of test tree trunks in different RPW infestation conditions to the simulated reflection response of test tree trunks in different RPW infestation conditions, the experimental reflection responses agree well with the simulated reflection responses. For example, the experimental resonant frequency of the healthy tree trunk and the simulated resonant frequency of the healthy tree trunk are both 0.95 GHz. The experimental resonant frequency of the partially damaged tree trunk and the simulated resonant frequency of the partially damaged tree trunk are 0.9 GHz and 0.89 GHz, respectively. The experimental resonant frequency of the completely damaged tree trunk and the simulated resonant frequency of the completely damaged tree trunk are 0.84 GHz and 0.85 GHz, respectively.

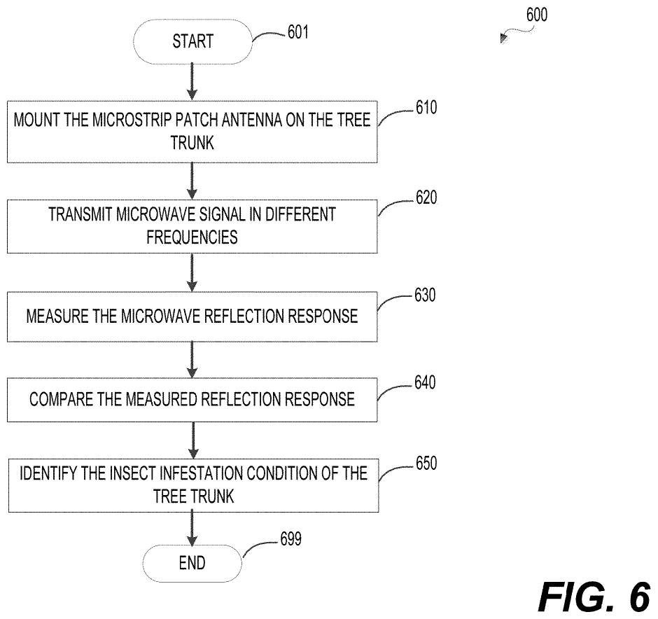

FIG. 6 is a flowchart outlining an exemplary process of detecting insect infestation according to an embodiment of the disclosure. The process 600 can be performed at the microstrip patch antenna 100. In the FIG. 6 example, the microstrip patch antenna 100 can be placed in contact with the test tree trunk 160. Herein, the tree trunk 160 can act as the superstrate of the microstrip patch antenna 100. The process can start from 601 and proceed to 610.

At 610, the microstrip patch antenna 100 can be mounted on a manually flattened surface of the test tree trunk 160, and the test tree trunk 160 can act as the superstrate of the microstrip patch antenna 100. Further, the cable 150 can connect the microstrip patch antenna 100 with a measurement device (e.g., an oscilloscope) 170 through the connector 130 and feed through the antenna GND plane 140. The process can then proceed to 620.

At 620, the microstrip patch antenna 100 can transmit microwave signals in different frequencies. The process can then proceed to 630.

At 630, the measurement device 170 can measure the microwave reflection response of the test tree trunk 160 at each frequency and stored the microwave reflection response in the storage medium 180. The process can then proceed to 640.

At 640, the processing circuitry 190 can compare the measured microwave reflection response of the test tree trunk 160 to the microwave reflection responses of one or more reference tree trunks. Herein, the microwave reflection responses of one or more reference tree trunks can be simulated the microwave reflection responses of one or more reference tree trunks in different insect infestation conditions, or/and the measured microwave reflection responses of one or more reference tree trunks whose different insect infestation conditions are known beforehand. The process can then proceed to 650.

At 650, the processing circuitry 190 can identify whether the test tree trunk is infected or not, and identify the insect infestation condition based on the comparison results. The process can then proceed to 699 and terminate.

Obviously, numerous modifications and Variations of the present invention are possible in light of the above teachings. It is therefore to be understood that within the scope of the appended claims, the invention may be practiced otherwise than as specifically described herein.

1. An apparatus for detecting insect infestation, comprising:
a microstrip patch antenna, configured to:
mount on a surface of a test tree trunk; and
transmit a microwave signal;
a measurement device, configured to:
measure a microwave reflection response of the test tree trunk in response to the microwave signal from the microstrip patch antenna;
a storage medium, configured to store:
the measured microwave reflection response of the test tree mink;
one or more microwave reflection responses of one or more reference tree trunks;
a program for comparing microwave reflection responses; and
a program for identifying an insect infestation condition of the test tree trunk; and
a processing circuitry, configured to:
read the stored microwave reflection responses;
compare the measured microwave reflection response of the test tree trunk to the microwave reflection responses of one or more reference tree trunks; and
identify the insect infestation condition of the test tree trunk.

2. The apparatus of claim 1, wherein the test tree trunk is a superstrate of the microstrip patch antenna.

3. The apparatus of claim 1, wherein the microstrip patch antenna configured to mount on, the surface of the test tree trunk, is further configured to place the circular patch of the microstrip patch antenna in contact with a manually flattened surface of the test tree trunk wherein the size of flattened surface is not smaller than the size of microstrip patch antenna.

4. The apparatus of claim 1, further comprising a plurality of microstrip patch antennas around the test tree trunk.

5. The apparatus of claim 1, wherein the microstrip patch antenna is configured to transmit the microwave signal from the microstrip patch antenna at a microwave signal frequency not above 1.2 GHz.

6. The apparatus of claim 1, wherein the storage medium is further configured to:
store one or more simulated microwave reflection responses of one or more reference tree trunks under different insect infestation conditions including healthy. partially damaged, or/and completely damaged; or
store one or more measured microwave reflection responses of one or more reference tree trunks under different insect infestation conditions including healthy, partially damaged, or/and completely damaged.

7. The apparatus of claim 1, wherein the processing circuitry, is further configured to:
load the program, stored in the storage medium, for comparing the measured microwave reflection response of the test tree trunk to the microwave reflection responses of the one or more reference tree trunks; and
execute the program to calculate the change between a resonant frequency of the test tree trunk and a resonant frequency of a healthy reference tree trunk, a partially damaged reference tree trunk, or/and a completely damaged reference tree trunk.

8. The apparatus of claim 1, wherein the processing circuitry, is further configured to:
identify the test tree trunk to be a healthy tree trunk when the change between a resonant frequency of the test tree trunk and a resonant frequency of a healthy reference tree trunk is below a threshold value;
identify the test tree trunk to be a partially damaged tree trunk when the change between a resonant frequency of the test tree trunk and a resonant frequency of a partially damaged reference tree trunk is below the threshold value; or
identify the test tree trunk to be a completed damaged tree trunk when the change between a resonant frequency of the test tree trunk and a resonant frequency of a completed damaged reference tree trunk is below the threshold value.

9. A method of detecting insect infestation in a test tree trunk, comprising:
mounting a microstrip patch antenna on a surface of the test tree trunk;
transmitting a microwave signal from the microstrip patch antenna;
measuring, by a measurement device, a microwave reflection response of the test tree trunk to the microwave signal;
storing, by a storage medium, the measured microwave reflection response of the test tree trunk, one or more microwave reflection responses of one or more reference tree trunks, a program for comparing microwave reflection responses, and a program for identifying an insect infestation condition of the test tree trunk; and
reading, by a processing circuitry, the stored microwave reflection responses;
comparing, by the processing circuitry, the measured microwave reflection response of the test tree trunk to the microwave reflection responses of one or more reference tree trunks; and
identifying, by the processing circuitry, the insect infestation condition of the test tree trunk.

10. The method of claim 9, wherein the test tree trunk is a super rate of the microstrip patch antenna.

11. The method of claim 9, wherein the mounting the microstrip patch antenna on the surface of the test tree trunk, comprises placing the circular patch of the microstrip patch antenna in contact with a manually flattened surface of the test tree trunk wherein the flattened surface is not smaller than the size of microstrip patch antenna.

12. The method of claim 9, wherein the mounting further comprises mounting a plurality of the microstrip patch antennas around the test tree trunk.

13. The method of claim 9, wherein the transmitting the microwave signal comprises exciting the test tree trunk with a microwave signal having a frequency not above 1.2 GHz.

14. The method of claim 9, wherein the storing the microwave reflection responses, further comprises:
storing one or more simulated microwave reflection responses of one or more reference tree trunks under different insect infestation conditions including healthy, partially damaged, or/and completely damaged; or
storing one or more measured microwave reflection responses of one or more reference tree trunks under different insect infestation conditions including healthy, partially damaged, or/and completely damaged.

15. The method of claim 9, wherein the comparing comprises:
loading the program, stored in the storage medium, for comparing the measured microwave reflection response of the test tree trunk to the microwave reflection responses of the one or more reference tree trunks; and
executing the program to calculate the change between a resonant frequency of the test tree trunk and a resonant frequency of a healthy reference tree trunk, a partially damaged reference tree trunk, or/and a completely damaged reference tree trunk.

16. The method of claim 9, wherein the identifying comprises:
identifying the test tree trunk to be a healthy tree trunk when the change between a resonant frequency of the test tree trunk and a resonant frequency of a healthy reference tree trunk is below a threshold value;
identifying the test tree trunk to be a partially damaged tree trunk when the change between a resonant frequency of the test tree trunk and a resonant frequency of a partially damaged reference tree trunk is below the threshold value; or
identifying the test tree trunk to be a completed damaged tree trunk when the change between a resonant frequency of the test tree trunk and a resonant frequency of a completed damaged reference tree trunk is below the threshold value.

17. A non-transitory computer readable medium storing instructions which, when executed by a processing circuitry, cause the processing circuitry to perform a method for:
reading stored microwave reflection responses;
comparing a measured microwave reflection response of a test tree trunk to micro e reflection responses of one or more reference tree trunks; and
identifying an insect infestation condition of the test tree trunk.

18. The non-transitory computer readable medium of claim 17, wherein the reading stored microwave reflection responses, further comprises:
reading a stored measured microwave reflection response of a test tree trunk; and
reading stored simulated microwave reflection responses of one or more reference tree trunks under different insect infestation conditions including healthy, partially damaged, or/and completely damaged; or
reading stored measured microwave reflection responses of one or more reference tree trunks under different insect infestation conditions including healthy, partially damaged, or/and completely damaged, wherein the insect infestation conditions are identified beforehand.

19. The non-transitory computer readable medium of claim 17, wherein the comparing the measured microwave reflection response of the test tree trunk to microwave reflection responses of one or more reference tree trunks, further comprises:
loading the program, stored in the storage medium, for comparing the measured microwave reflection response of the test tree trunk to the microwave reflection responses of the one or more reference tree trunks: and
executing the program to calculate the change between a resonant frequency of the test tree trunk and a resonant frequency of a healthy reference tree trunk, a partially damaged reference tree trunk, or/and a completely damaged reference tree trunk.

20. The non-transitory computer readable medium of claim 17, wherein the identifying the insect infestation condition of the test tree trunk, further comprises:
identifying the test tree trunk to be a healthy tree trunk when the change between a resonant frequency of the test tree trunk and a resonant frequency of a healthy reference tree trunk is below a threshold value;
identifying the test tree trunk to be a partially damaged tree trunk when the change between a resonant frequency of the test tree trunk and a resonant frequency of a partially damaged reference tree trunk is below the threshold value; or
identifying the test tree trunk to be a completed damaged tree trunk when the change between a resonant frequency of the test tree trunk and a resonant frequency of a completed damaged reference tree trunk is below the threshold value.
